(19) United States
(12) Patent Application Publication
(10) Pub. No.: US 2024/0016979 A1
(43) Pub. Date: Jan. 18, 2024

(54) AMNIOTIC MEMBRANE FOR MYOCARDIAL REPAIR

(71) Applicant: The Regents of the University of California, Oakland, CA (US)

(72) Inventors: Song Li, Beverly Hills, CA (US); Randall J. Lee, Hillsborough, CA (US); Jeff Henry, Los Angeles, CA (US)

(73) Assignee: The Regents of the University of California, Oakland, CA (US)

(21) Appl. No.: 18/255,307

(22) PCT Filed: Dec. 22, 2021
(86) PCT No.: PCT/US21/64825

§ 371 Date: May 31, 2023

Related U.S. Application Data
(60) Provisional application No. 63/129,013, filed on Dec. 22, 2020.

(51) Int. Cl. A61L 27/36; A61L 27/52; A61L 27/54; A61L 27/48

(52) U.S. Cl. CPC A61L 27/3604; A61L 27/3691; A61L 27/3687; A61L 27/52; A61L 27/54; A61L 27/367; A61L 27/48; A61L 2400/06; A61L 2430/20; A61L 2300/41

(57) ABSTRACT

A novel injectable human amniotic membrane (hAM) matrix to enhance cardiac repair and/or regeneration following myocardial injury (MI) and wound healing has been developed. The invention disclosed herein provides human amniotic membranes isolated from human placenta and engineered to be a thermo-responsible, injectable gel at temperature ranges that fall within body temperature. The ultrasound-guided injection of hAM matrix into a rodent model of myocardial infarction significantly improved cardiac contractility, as measured by ejection fraction (EF), and decreased fibrosis. The disclosure provided herein demonstrates the specific engineering injectable hAM matrices and their efficacy in attenuating degenerative changes in cardiac function following MI, which has broad applications in wound healing and tissue regeneration.

### CROSS-REFERENCE TO RELATED APPLICATIONS

This application claims the benefit under 35 U.S.C. Section 119(e) of co-pending and commonly-assigned U.S. Provisional Patent Application Ser. No. 63/129,013, filed on Dec. 22, 2020, and entitled “AMNIOTIC MEMBRANE FOR MYOCARDIAL REPAIR” which application is incorporated by reference herein.

### STATEMENT REGARDING FEDERALLY SPONSORED RESEARCH AND DEVELOPMENT

This invention was made with government support under Grant Numbers HL117213, HL121450 and HL094162, awarded by the National Institutes of Health.

The government has certain rights in the invention.

### TECHNICAL FIELD

Embodiments of the disclosure concern at least the fields of cardiology and medicine.

### BACKGROUND OF THE INVENTION

Myocardial infarction (MI) associated heart failure is the leading cause of death in the U.S.⁽¹⁾ MI occurs when blood flow to the heart from coronary arteries is occluded, causing ischemia and subsequent myocardial tissue death. Myocardial tissue is unable to effectively regenerate following MI, thus leading to scar formation, left ventricular remodeling, and eventual heart failure.^{(2,3)} Several tissue engineering strategies have been developed to prevent scarring and promote cardiac regeneration, including cell-based therapies, porous scaffolds, cardiac patches and hydrogels.⁽⁴⁻⁹⁾ Among them, an injectable natural or synthetic hydrogel is minimally invasive and promising for in situ cardiac tissue repair for infarcted hearts.⁽¹⁰⁾

Decellularized mammalian Extracellular matrix (ECM), as a natural material, has been extensively investigated in regenerative medicine.⁽¹¹⁻¹⁸⁾ ECM hydrogel retains the full biochemical complexity and inherent bioactivity of the native matrix, which could facilitate tissue regenerative capability.^{(9, 19, 20)} Although certain properties of ECM hydrogels are widely conserved regardless of source tissue, some characteristics vary markedly and are influenced by source species, source tissue and processing methods. In particular, human amniotic membrane (hAM) can be easily obtained and processed from the placenta without ethical concerns. The matrix derived from human amniotic membrane has various components and bioactivities, which allow such matrix to be widely applied in corneal transplantation, retinal regeneration, liver regeneration, and wound healing.⁽²¹⁻²⁴⁾ Due to its anti-inflammatory effects, anti-fibrotic effect, and angiogenic potentials,⁽²⁵⁻²⁸⁾ materials derived from hAM could beneficial for restoring cardiac function following MI.

Methods and materials that can utilize injectable hydrogel matrices to restore cardiac function following myocardial infarction are needed. Embodiments of the invention meet this need.

### SUMMARY OF THE INVENTION

As discussed below, we have developed an injectable human amniotic membrane (hAM) matrix that enhances cardiac regeneration following myocardial injury (MI). The invention disclosed herein provides human amniotic membranes isolated from human placenta and engineered to form a thermo-responsible, injectable hydrogel at temperature ranges that fall within human body temperature. Embodiments of the invention include such amniotic membrane hydrogel matrix compositions and methods of making and using them. The disclosure provided herein teaches the engineering of these injectable hAM matrices and then their efficacy in promoting wound healing and tissue regeneration. Embodiments of the invention are useful for attenuating degenerative changes in cardiac function following MI, material properties which have broad applications in tissue regeneration.

The invention disclosed herein has a number of embodiments. Embodiments of the invention include, for example, methods of making human amniotic membrane hydrogel matrix compositions. Typically, these methods comprise combining a human amniotic membrane with agents selected to remove cells and nucleic acids from the human amniotic membrane; and then processing the human amniotic membrane hydrogel matrix so as to form either a powder, or alternatively a liquid/gel composition that is suitable for in vivo use. These methods can include, for example, rinsing and/or dialyzing the decellularizing human amniotic membrane so as to remove agents selected to remove cells and nucleic acids; lyophilizing the rinsed and/or dialyzed decellularized human amniotic membrane; forming a dry powder from the lyophilized human amniotic membrane so that a powder composition is formed. Typically, the methods further include solubilizing this dry powder to form a solution; adjusting the pH of the solubilized dry powder solution; and then lyophilizing the pH adjusted solubilized dry powder solution, so that the human amniotic membrane hydrogel matrix powder composition is formed. Typically in these methods, the human amniotic membrane hydrogel matrix composition is formed using methodological steps and or reagents selected so that the glycosoaminoglycan content of the human amniotic membrane hydrogel matrix composition is at least 80% (e.g. about 85%) of the glycosoaminoglycan content of the native human amniotic membrane; and/or the human amniotic membrane hydrogel matrix composition exhibits a collagen content that is at least 40% or (e.g. about 45%) of the collagen content of the native human amniotic membrane.

Typically, the methods of making the human amniotic membrane hydrogel matrix composition further comprise resuspending the human amniotic membrane hydrogel matrix powder composition in an aqueous solution so as to form a liquid/gel injectable human amniotic membrane hydrogel matrix. In illustrative embodiments of the invention, the composition forms a thermo-responsible, injectable hydrogel at temperature ranges that fall within body temperature. Typically, for example, the injectable human amniotic membrane hydrogel matrix composition is designed to form a viscous liquid at temperatures from 0° C. to 20° C.; and to form a gel at 37° C. In embodiments of the invention, the injectable human amniotic membrane hydrogel matrix composition can further comprise additional constituents such as crosslinking agents, and/or pharmaceutical excipients such as those selected from the group consisting of: a preservative, a tonicity adjusting agent, a detergent, a viscosity adjusting agent, a sugar and a pH adjusting agent; and/or a therapeutic agent such as a wound healing agent, an anti-fibrotic agent, an anti-inflammatory agent, a hemostatic agent, or a chemotherapeutic agent.

Embodiments of the invention include compositions formed by the methods disclosed herein. Embodiment of the invention include, for example, compositions of matter comprising a human amniotic membrane hydrogel matrix, wherein the human amniotic membrane hydrogel matrix is decellularized, and is in either a powder form, or is in solution. Typically, in these embodiments, the human amniotic membrane hydrogel matrix comprises a glycosoaminoglycan content that is at least 80% of the glycosoaminoglycan content of a native human amniotic membrane; and/or the human amniotic membrane hydrogel matrix comprises a collagen content that is at least 45% of the collagen content of the native human amniotic membrane; and/or at least 95% of native DNA in the human amniotic membrane hydrogel matrix has been removed. In certain embodiments of the invention, the hAM hydrogel gel exhibits certain selected material properties, for example at 37° C., the composition exhibits a shear modulus of 1 PA-20 kPA, for example about 5 PA to about 10 PA (e.g., about 7.5±2.4 PA) at a frequency of about 1 Hz. Typically, the injectable human amniotic membrane hydrogel matrix compositions form a viscous liquid at temperatures from 0° C. to 20° C.; and form a gel at 37° C.

Yet another embodiment of the invention is a method of delivering a liquid/gel hAM composition disclosed herein to a preselected site such as a site or trauma or injury in vivo. These method comprise: disposing the composition in the form of a liquid (typically at a temperature between 0° C. to 20° C.) in a vessel having a first end comprising an opening and a second end; applying a force to the second end of the vessel, wherein the force is sufficient to force the liquid through the first end of the vessel; and delivering the composition out of the vessel through the opening and to the preselected site (e.g. a site having a temperature of about 37° C. where the liquid composition will then form a gel). In typical embodiments of the invention, the site is at an in vivo location, for example one where an individual has experienced cardiac injury.

Related embodiments of the invention include methods of using the hAM compositions disclosed herein to promote wound healing and tissue regeneration (e.g., cardiac regeneration). For example, embodiments of the invention include methods of inhibiting fibrosis at a site of cardiac injury in an individual, the methods comprising: disposing a liquid/gel hAM composition disclosed herein at the site of the cardiac injury such that the composition modulates cardiac remodeling, so that fibrosis is inhibited.

In certain embodiments of the invention, the modulation of cardiac remodeling comprises an inhibition of negative ventricular remodeling; and/or a decrease in myocardial infarction size. Optionally in these methods, the composition further comprises additional constituents such a pharmaceutical excipient, a therapeutic agent and/or human cells.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

The figures show illustrative aspects and embodiments of the invention.

FIG. 1. Physical appearance of hAM matrix during various stage of processing. (A) Untreated hAM prior to decellularization. (B) Decelllarized hAM tissue. (C) Lyophilized and ground soluble hAM matrix. (D) Gelation of decellularized hAM matrix at 37° C. (E) Rheological measurement for storage modulus of hAM matrix between oscillatory frequencies 10 and 0.1 Hz at 1% strain amplitude. Data presented as a mean of 5 separate measurements±one standard deviation.

FIG. 2. Histological and biochemical characterization of CHAPS and SDS decellularized hAM tissue. (A-C) Representative H&E staining images of untreated, CHAPS decellularized and SDS decellularized hAM. (D-F) DAPI staining of nuclei present in untreated, CHAPS decellularized, and SDS decellularized hAM. (G) Percentage of DNA content remaining after CHAPS or SDS decellularization. (H) Percentage of glycosoaminoglycan (GAG) content remaining after CHAPS or SDS decellularization. (1) Percentage of collagen content remaining after CHAPS or SDS decellularization.

FIG. 3: Proliferation, biocompatibility, viability of bovine aortic endothelial cells on hAM matrix. BAECs were seeded at low (1000 cells/cm²), medium (5000 cells/cm²), and high density (10,000 cells/cm²) on either hAM matrix or collagen-1. Proliferation was measured using EdU (A) and is presented as the percentage of total DAPI stained cells expressing positive EdU staining (B). (C) Live/dead staining of ECs at days 2 and 6. Cells were seeded at 5000 cells/cm². (D) Cell viability assay at days 2 and 6. Cells were seeded at 5000 cells/cm².

FIG. 4: Effect of hAM matrix injection on LV ejection fraction (LVEF) and LV fractional shortening (LVFS) after 5 weeks following acute MI. (A) Average LVEF before and after injection of PBS or hAM matrix, labeled as pre-treatment (Pre) or post-treatment (Post) respectively. (B) LVEF of each animal before and after injection of PBS or hAM matrix. (C) Average of LVEF changes by using pre-treatment value of each animal as a reference. (D) Average of LVFS changes by using pre-treatment value in each animal as a reference. n=5 for each group, * p<0.05.

FIG. 5. hAM matrix promotes adult cardiac regeneration after MI. (A) Representative images of Masson trichrome-stained heart sections at 5 weeks after hAM matrix or PBS injection. Scale bars represent 4 mm. (B) Measurement of infarcted size at 5 weeks after hAM matrix or PBS injection analyzed by Masson trichrome staining. n=5 for each group, * p<0.05.

### DETAILED DESCRIPTION OF THE INVENTION

In the description of embodiments, reference may be made to the figures which form a part hereof, and in which is shown by way of illustration a specific embodiment in which the invention may be practiced. It is to be understood that other embodiments may be utilized, and structural changes may be made without departing from the scope of the present invention.

Ischaemic heart disease represents the leading cause of death worldwide. Heart failure following myocardial infarction (MI) is associated with severe fibrosis formation and cardiac remodeling. Recently, injectable hydrogels have emerged as a promising approach to repair the infarcted heart and improve heart function through minimally invasive administration. As discussed below, we have developed a novel injectable human amniotic membrane (hAM) matrix to enhance cardiac regeneration following MI. Briefly, human amniotic membrane was isolated from human placenta and engineered to form a thermo-responsive, injectable gel around body temperature. Ultrasound-guided injection of the hAM matrices of the invention into rat MI hearts significantly improved cardiac contractility, as measured by ejection fraction (EF), and decreased fibrosis. These results demonstrate the feasibility of engineering an injectable hAM matrix and its efficacy in attenuating degenerative changes in cardiac function following MI, material properties which have broad applications in tissue regeneration.

Here we report methods for making and using a decellularized hAM matrix. In vitro cell culture shows that cell proliferation and other biological activities of the hAM matrices disclosed herein are preserved. Additionally, as disclosed herein, when a thermo-responsive hAM matrix hydrogel is injected into the rat MI hearts, this composition demonstrates a number of beneficial effects, for example the enhancement of cardiac ejection fraction as well as the reduction of fibrosis.

Embodiments of the invention include, for example, methods of making human amniotic membrane hydrogel matrix compositions. Such methods typically comprise combining a human amniotic membrane with agents selected to remove cells and nucleic acids from the human amniotic membrane; and then processing the human amniotic membrane hydrogel matrix so as to form either a powder, or alternatively a liquid/gel composition that is suitable for in vivo use. These methods can include, for example, rinsing and/or dialyzing the decellularizing human amniotic membrane so as to remove agents selected to remove cells and nucleic acids; lyophilizing the rinsed and/or dialyzed decellularized human amniotic membrane; forming a dry powder from the lyophilized human amniotic membrane so that a dry powder composition is formed. Typically, the methods further include solubilizing this dry powder to form a solution; adjusting the pH of the solubilized dry powder solution; and then lyophilizing the pH adjusted solubilized dry powder solution, so that the human amniotic membrane hydrogel matrix powder composition is formed. Typically in these methods, the human amniotic membrane hydrogel matrix composition is formed using methodological steps and or reagents selected so that the glycosoaminoglycan content of the human amniotic membrane hydrogel matrix composition is at least 10%, 20%, 30%, 40% 50%, 60%, 70% or 80% of the glycosoaminoglycan content of the native human amniotic membrane; and/or the human amniotic membrane hydrogel matrix composition exhibits a collagen content that is at least 10%, 20%, 30%, 40% or 45% of the collagen content of the native human amniotic membrane. In certain embodiments of the invention, the human amniotic membrane hydrogel matrix comprises a glycosoaminoglycan content that is not more than 90% or 95% of the glycosoaminoglycan content of a native human amniotic membrane. In some embodiments of the invention, the human amniotic membrane hydrogel matrix comprises a collagen content that is not more than 50% or 60% of the collagen content of the native human amniotic membrane.

Typically, the methods of making the human amniotic membrane hydrogel matrix composition further comprise resuspending the human amniotic membrane hydrogel matrix powder composition in an aqueous solution so as to form a liquid/gel injectable human amniotic membrane hydrogel matrix. In illustrative embodiments of the invention, the composition forms a thermo-responsible, injectable hydrogel at temperature ranges that fall within body temperature. Typically, for example, the injectable human amniotic membrane hydrogel matrix composition is designed to form a viscous liquid at temperatures from 0° C. to 20° C.; and to form a gel at 37° C. In embodiments of the invention, the injectable human amniotic membrane hydrogel matrix composition can further comprise additional constituents such as crosslinking agents, and/or pharmaceutical excipients such as those selected from the group consisting of a preservative, a tonicity adjusting agent, a detergent, a viscosity adjusting agent, a sugar and a pH adjusting agent; and/or a therapeutic agent such as a wound healing agent, an anti-fibrotic agent, an anti-inflammatory agent, a hemostatic agent, or a chemotherapeutic agent; and/or mammalian cells.

Embodiments of the invention include compositions formed by the methods disclosed herein. Embodiment of the invention include, for example, compositions of matter comprising a human amniotic membrane hydrogel matrix, wherein the human amniotic membrane hydrogel matrix is decellularized, and is in either a powder form, or is in solution. Typically in these embodiments, the human amniotic membrane hydrogel matrix comprises a glycosoaminoglycan content that is at least 10%, 20%, 30%, 40% 50%, 60%, 70% or 80% of the glycosoaminoglycan content of a native human amniotic membrane; and/or the human amniotic membrane hydrogel matrix comprises a collagen content that is at least 10%, 20%, 30%, 40% or 45% of the collagen content of the native human amniotic membrane; and/or at least 85%-95% of native DNA in the human amniotic membrane hydrogel matrix has been removed.

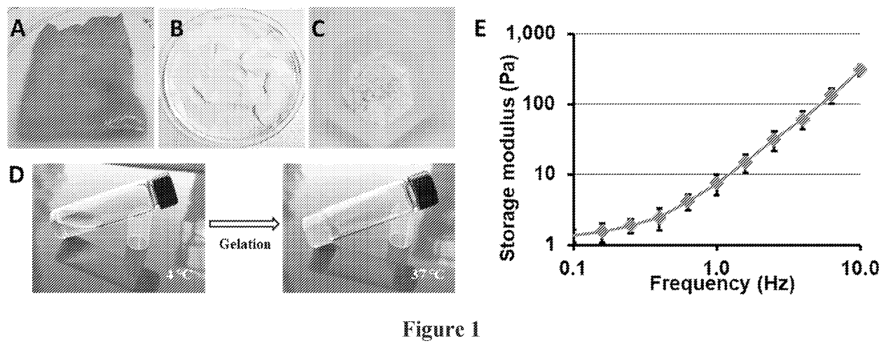

Certain embodiments of the invention comprise a (hAM) hydrogel composition, wherein: the human amniotic membrane comprises a glycosoaminoglycan content comprising from about 70% to about 90% of the glycosoaminoglycan content found in native human amniotic membranes; the human amniotic membrane comprises a collagen content comprising from about 30% to about 60% of the collagen content found in native human amniotic membranes; and the human amniotic membrane comprises less than 5% or less than 10% of the DNA content found in native human amniotic membranes. Such human amniotic membrane hydrogel compositions of the invention have a number of unexpected and desirable material properties including, for example, an ability to form a viscous liquid at temperatures from 0° C. to 20° C.; and to form a gel at 37° C. In certain embodiments of the invention, such hAM hydrogel compositions exhibit other desirable material properties, including for example at 37° C., a shear modulus of 1 PA-20 kPA, for example about 5 PA to about 10 PA (e.g., about 7.5±2.4 PA) at a frequency of about 1 Hz (see, e.g., FIG. 1E).

Yet another embodiment of the invention is a method of delivering a liquid/gel hAM composition disclosed herein to a preselected site such as a site or trauma or injury in vivo, the method comprising: disposing the composition in the form of a liquid (typically at a temperature between 0° C. to 20° C.) in a vessel having a first end comprising an opening and a second end; applying a force to the second end of the vessel, wherein the force is sufficient to force the liquid through the first end of the vessel; and delivering the composition out of the vessel through the opening and to the preselected site (e.g. a site having a temperature of about 37° C. where the liquid composition will then form a gel). In typical embodiments of the invention, the vessel is a catheter, and the site is at an in vivo location, for example one where an individual has experienced cardiac injury.

Related embodiments of the invention include methods of using the hAM compositions disclosed herein to promote wound healing and tissue regeneration (e.g., cardiac regeneration). For example, embodiments of the invention include methods of inhibiting fibrosis at a site of cardiac injury in an individual, the methods comprising: disposing a liquid/gel hAM composition disclosed herein at the site of the cardiac injury such that the composition modulates cardiac remodeling, so that fibrosis is inhibited. In certain embodiments of the invention, the modulation of cardiac remodeling comprises an inhibition of negative ventricular remodeling; and/or a decrease in myocardial infarction size. Optionally in these methods, the composition further comprises additional constituents such a therapeutic agent and/or human cells.

In embodiments of the invention, the injectable human amniotic membrane hydrogel matrix composition can further comprise additional constituents such as crosslinking agents (e.g. citric acid derivatives and the like), and/or pharmaceutical excipients such as those selected from the group consisting of: a preservative, a tonicity adjusting agent, a detergent, a viscosity adjusting agent, a sugar and a pH adjusting agent; and/or a therapeutic agent such as a wound healing agent, an anti-fibrotic agent, an anti-inflammatory agent, a hemostatic agent, or a chemotherapeutic agent; and/or mammalian cells (e.g. lymphocytes or stem cells). For compositions suitable for administration to humans, the term “excipient” is meant to include, but is not limited to, those ingredients described in Remington: The Science and Practice of Pharmacy, Lippincott Williams & Wilkins, 21st ed. (2006) (hereinafter Remington's). In addition, the constituents of the compositions, their relative concentrations, and certain methodological steps can be selected to modulate hydrogel characteristics such as the gelation dynamics of the compositions of the invention (e.g., the relative concentrations of hAM within the compositions, the amounts of crosslinking agents within the compositions, the temperature of the compositions and the like).

Further aspects and embodiments of the invention are disclosed in the following sections.

### Methods

Decellularization of Human Amniotic Membrane (hAM)

Full term human placentas with intact hAM were obtained from healthy donors undergoing caesarean section at the University of California, San Francisco or University of California, Davis Hospitals. The hAM was then identified and separated from the placenta and weighed. The tissue was subsequently frozen at −80° C. for 48 hours and thawed prior to decellularization. The membrane was thoroughly rinsed with water and soaked in 1 N NaCl for 15 minutes. Membranes were then treated in a solution containing 8 mM CHAPS (3-((3-cholamidopropyl) dimethylammonio)-1-propanesulfonate), 25 mM EDTA, and 1M NaCl at room temperature with moderate agitation. The CHAPS solution was removed and replenished every 2 hours, for 6 hours total. For comparison purposes, 8 mM SDS (sodium dodecyl sulfate) was used instead of CHAPS. Following CHAPS or SDS treatment, all membranes were subsequently washed with Tris-buffered saline (TBS, 3 times, 20 minutes each). To remove remaining nucleic acid material, membranes were incubated in a 90 U/ml benzonase solution (Sigma Aldrich Inc., St. Louis, MO) for 15 hours at 37° C. To enhance nuclease activity, benzonase was prepared in 50 mM Tris-HCL pH 8.0, with 0.1 mg/ml BSA, and 1 mM MgCl₂. Following benzonase treatment, hAMs were washed repeatedly in TBS, followed by repeated washes with distilled water. hAMs were further decellularized and disinfected using a 0.1% peracetic acid, 4% ethanol and water. After repeated washing in TBS, and distilled water, hAMs were dialyzed against water at 4° C. for 4 days to remove excess reagents.

Following dialysis, portions of the decellularized hAM were snap-frozen in OCT compound (Sakura Finetek, Torrance, CA) and sectioned for histological analysis. Intact and untreated hAM was also frozen and sectioned for comparison. Sections were stained with DAPI or Hematoxylin & Eosin to visualize nuclei and matrix components respectively.

### Preparation of Injectable hAM Matrix

Decellularized hAMs were lyophilized overnight and subsequently ground into a dry powder using a motorized grinder. The decellularized hAM powder was solubilized by digestion in 0.2 mg/ml pepsin and 0.1N HCL for 48 hours at room temperature. A ratio of 20 mg hAM matrix per 1 ml pepsin/HCL solution was used. After 48 hours, the solution pH was adjusted to 8.0 using 10 N NaOH and 10×PBS. The pH adjusted matrix was then re-lyophilized overnight, ground into powder form, and stored at 4° C. Prior to experiments, the hAM matrix was sterilized in ethylene oxide gas overnight. To induce gelation, hAM matrix was dissolved in PBS (20 mg/ml) and allowed to gel at 37° C.

### Characterization of Injectable hAM Matrix

The remaining DNA content in the injectable hAM matrix was quantified using a Quant-iT™ PicoGreen® Assay (Invitrogen, Grand Island, NY). A DNeasy Blood & Tissue Kit® (Qiagen, Valencia, CA) was used to isolate DNA prior to quantification using the assay. The DNA content of CHAPS processed hAM matrix, and SDS processed hAM matrix is presented as a percentage of native, untreated hAM tissue.

Glycosaminoglycans (GAGs) are often reported to be lost following decellularization of tissue. For this reason, we quantified the GAG content of both CHAPS processed hAM matrix and SDS processed hAM matrix. GAG content was quantified using a colorimetric 1,9 dimethylmethylene blue (DMMB) assay as described previously.⁽²⁹⁾ This reagent specifically binds to the sulfate and carboxyl groups of sulfated GAGs to cause a metachromatic shift. This shift in absorption can then be quantified via spectroscopy at 530 nm. Lyophilized powder native and decellularized hAM tissue was first digested using proteinase K (1 mg proteinase K/80 mg hAM matrix). DMMB reagent (16 μg/mL) was then allowed to react with the samples. GAG content in each sample was then immediately quantified by measuring the absorbance at 530 nm and comparing to values obtained using known GAG concentrations. Final GAG content is given as a percentage of GAG content in the native tissue. The hAM matrix processed using CHAPS contained the highest amount of preserved GAG content. For this reason, the CHAPS hAM matrix was used in all in vitro and in vivo experiments.

The collagen content of the decellularized hAM matrix was measured by a hydroxyproline assay kit (Cell Biolabs Inc) by following the vendor's protocol as previously described.⁽³⁰⁾ Briefly, hAM powder was hydrolyzed by 12N hydrochloric acid for 3 hours at 120° C. to free hydroxyproline. The acid-hydrolyzed samples were further dried under vacuum evaporation at 80° C. for 40 minutes. Then chloramine T mixture was added to convert the hydroxyproline to a pyrrole. Finally, Ehrlichs Reagent was added to the solution to react with the pyrrole and produce a chromophore. The absorbance of the samples was measured at 540 nm and quantified by a standard curve of hydroxyproline. The collagen content was calculated by using the estimation that hydroxyproline makes up 13.5% of collagen.

Mass spectrometry was performed in order to survey the overall protein composition of the injectable hAM matrix. Samples were digested with trypsin and desalted with C18 spec tips prior to analysis. Proteins were separated and analyzed at the Vincent J. Coates Proteomics/Mass Spectrometry Laboratory by using multidimensional protein identification technology (MudPIT) with two-dimensional HPLC separation-tandem mass spectrometry. Peptide identification and data analysis were performed using SEQUEST.

### Mechanical Characterization of Injectable hAM Matrix

Oscillatory rheology analysis was performed on a MCR 301 rheometer (Anton Paar). hAM matrix (400 μl) was allowed to gel for 1 hr at 37° C. on the rheometer between parallel plates with a 25-mm top probe at 0.81 mm gap width in a humidified chamber. Frequency sweep was performed from 10-0.1 Hz at 1% strain amplitude. Shear modulus was reported as the average of 5 storage modulus measurements taken at 1 Hz.

In vitro Characterization of Cell Growth on Injectable hAM Matrix

In vitro biocompatibility was examined by investigating the effect of injectable hAM matrix on the proliferation of bovine arterial endothelial cells (BAECs). BAECs were harvested from fresh bovine aortas as previously described using collagenase and gentle scrapping with a policeman.⁽³¹⁾ All cells were cultured in Dulbecco's Modified Eagle Medium (DMEM) supplemented with 10% fetal bovine serum (FBS) and 1% penicillin/streptomycin antibiotic mix. Cell culture was maintained in a humidified incubator at 37° C.

For proliferation experiments, the surfaces of a 24-well tissue culture plate were coated with either 1 mg/ml rat tail collagen type-1 (BD Biosciences) or 5 mg/ml injectable hAM matrix. BAEC were seeded on coated surfaces at low density (1000 cells/cm²), medium density (5,000 cells/cm²), and high density (10,000 cells/cm²). After 48 hours, BAEC proliferation on hAM matrix and collagen type-1 surfaces was then measured and compared using a Click-iT® EdU Alexa Fluor® 488 Detection Kit (Life Technologies Inc). Samples were counterstained using DAPI. Cell proliferation is given as the percent of EdU positive cells.

In addition, cell biocompatibility and viability were evaluated. Live/dead staining (Live/Dead staining Kit, Thermo Fisher Scientific) was employed to show the cell biocompatibility on either hAM matrix or collagen-I at days 2 and 6. ECs were seeded at 5000 cells/cm². Fluorescent images were taken using Zeiss Axio Observer Z1 inverted microscope to visualize stained cells. Furthermore, the cell viability was quantitatively measured by PrestoBlue® assay (Thermo Fisher Scientific).

### Myocardial Infarction (MI) Model

All surgical procedures were approved by the Committee for Animal Research of the University of California, San Francisco. The ischemia-reperfusion model used in this study has been previously used extensively as a model for MI.^{(32,33)} Female Sprague-Dawley rats (225-250 g) were anesthetized with ketamine (90 mg/kg) and xylazine (10 mg/kg). The chest was opened by a median sternotomy, and a single stitch of 7-0 Ticron suture (Tyco Healthcare, Norwalk, CT) was introduced around the left anterior descending (LAD) coronary artery and tightened to occlude for 30 min before reperfusion and closing the thoracic cavity. The animals were allowed to recover for 2 days after ischemia-reperfusion prior to treatment.

In vivo hAM Matrix Injection Studies

At 2 days post-MI, rats in the study group were randomized into hAM matrix (n=5) or PBS control groups (n=5). The animals were anesthetized using 2% isoflurane at 2 liters/minute and positioned on the echo-guided ultrasound apparatus. Echocardiography was performed prior to injection. Echocardiography directed intramyocardial injection of either 500 μL of sterile phosphate buffered saline (PBS) (UCSF Cell Culture Facility, San Francisco, CA) containing no hAM or 10 mg of hAM using a 27-gauge needle was delivered to the superior pole, center, and inferior pole of the infarct region as visualized by hyper-echoicity on ultrasound as previously described (Vevo 660, Visual Sonics, Toronto, Canada).⁽³⁴⁾ The successful injection was confirmed visually by changes in ultrasound signal in the vicinity of the syringe.

Functional Recovery of LV function after hAM Matrix Injection

Transthoracic echocardiography was performed immediately prior to injection, and 5 weeks after injection, Echocardiograms were used to calculate ejection fraction and fractional shortening before and after treatment. Ejection fraction was calculated as described previously.⁽³⁵⁾ A parasternal long-axis B-mode image was acquired in order to identify the maximum LV length. Three short-axis B-mode images were also acquired at basal, midventricular, and apical LV levels. Results are presented as the percent change in ejection fraction and percent change in fractional shortening. Using frames from the long axis images, as well as maximum and minimum cross-sectional areas in the heart cycle, LV end-systolic volume (LVESV) and LV end-diastolic volume were calculated. LV Ejection Fraction (LVEF) was calculated as follows, LVEF=((LVEDV−LVESV)/LVEDV)×100. Results are given as the percent change in LVEF 5 weeks post-injection. Left ventricular inner dimensions at systole (LVIDs) and diastole (LVIDd) were calculated using the leading-edge method, as prescribed by the American Society of Echocardiography. Fractional shortening, FS was calculated as follows, ((LVIDd−LVIDs)/LVIDd)×100. Results are presented as the percent change in FS 5 weeks post-injection.

### Histology

Five weeks post-treatment, the rats perfused with 3M KCl prior to sacrifice to arrest the hearts in the diastolic phase. The hearts were harvested, rinsed in cold saline, blotted-dry and fresh frozen in Tissue Tek O.C.T. freezing medium (Sakura Finetek, Torrance, CA). The hearts were sectioned into 10 μm slices. Sequential slides spanning the LV region were stained with Masson's-trichrome stain and were used for morphological assessment of the infarct size as previously described.⁽³⁶⁾ Briefly, Masson's-trichrome staining images were used to evaluate the infarct size with Image J software. The infarct size (% LV) was calculated by dividing the collagen deposited area to the entire left ventricle area near the midsection of the infarct area. Three sections for each sample were measured.

### Statistics

All experiments are performed in triplicate unless otherwise mentioned. In vitro and in vivo data are presented as mean±standard deviation. All data were compared with one-way ANOVA tests. Holm's t-test was performed to evaluate significant differences between pairs. A p-value of less than 0.05 was considered statistically significant.

### Results

Characterization of Injectable Human Amniotic Membrane (hAM) Matrix

An injectable hAM matrix was prepared through decellularization of hAM tissue (FIG. 1A&B). Using a series of chemical treatments, which included the use of either CHAPS or SDS detergent, hAM tissue was successfully decellularized, and lyophilized into power (FIG. 1C). The solubilized hAM matrix remained a viscous liquid while on ice or at room temperature. Gelation could be induced when placed at 37° C., as the resulting material became a soft gel that required gentle handling (FIG. 1D). The hAM gel had a shear modulus of 7.5±2.4 Pa as determined by oscillatory rheometry.

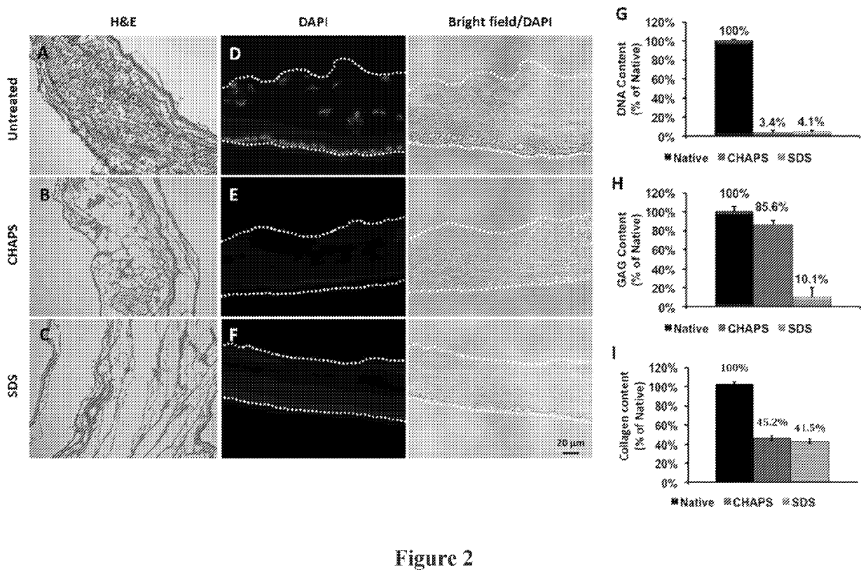

The decellularized tissue was sectioned and stained for nuclei using either DAPI or hematoxylin & eosin (H&E) stain. Both CHAPS and SDS decellularization processes were successful, as both tissues groups appeared devoid of cells (FIG. 2). The general matrix structure of native, CHAPS decellularized and SDS decellularized hAM was visualized using H&E staining (FIG. 2 A-C). The matrix structure of CHAPS decellularized hAM appears to be minimally altered in comparison to native hAM. In contrast, the matrix structure of SDS decellularized hAM appears significantly altered when compared to the structure of native hAM, DAPI staining (FIG. 2 D-F) showed that cell nuclei were removed by either CHAPS or SDS treatment.

In order to quantify the efficiency of decellularization protocols, remaining DNA content within the decellularized hAM matrix was quantified (FIG. 2G). The amount of DNA/mg of both SDS and CHAPS processed tissue was measured as a percentage of the amount of DNA/mg of untreated hAM tissue. Following CHAPS and SDS decellularization, little DNA content was detected. GAGs are a critical component of the extracellular matrix and also are a component commonly lost after decellularization. For this reason, the GAG content within CHAPS and SDS decellularized hAM was measured (FIG. 211). GAG content was measured as a percentage of the GAG content found within native, untreated hAM. After CHAPS decellularization, GAGs were relatively conserved, as 85.6% of the native GAG in hAM was retained. In contrast, however, only 10.1% of native GAG content was retained following decellularization using SDS. In addition, collagens are most abundant matrix proteins in the animal or human tissues. We found that more than 45% collagens were retained after the CHAPS decellularization process, which was slightly higher than SDS decellularization method (˜—41%). Since effective decellularization could be achieved using CH-APS with higher GAG/matrix components and collagen content, CHAPS-processed hAM was used in further in vitro and in viva experiments.

The composition of the injectable hAM matrix was characterized by the mass spectrometry analysis (Table 1). This identified and confirmed some major matrix components, including different types of collagen, fibronectin, laminin, keratin, fibrillin and growth factor receptors.

### In Vitro Studies Using Bovine Aortic Endothelial Cells (BAECs)

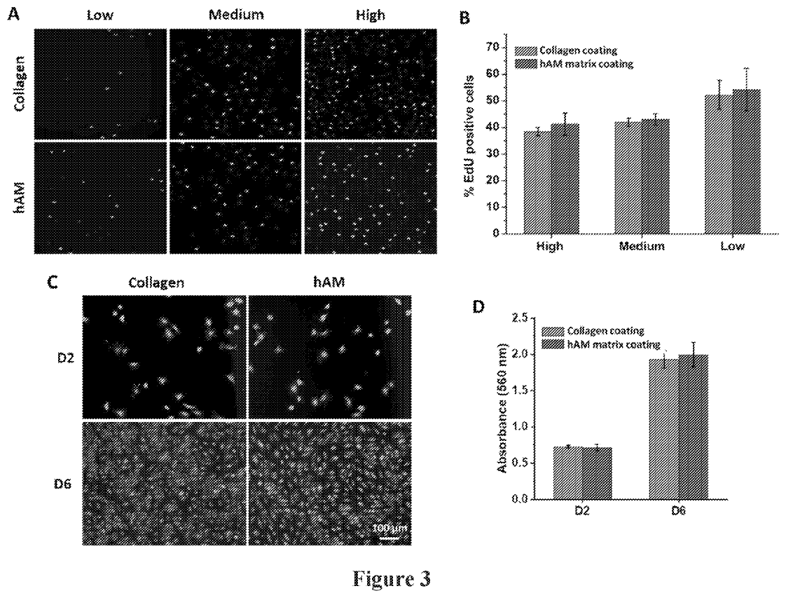

To assess the biocompatibility of the injectable hAM matrix, the proliferation of BAECs on hAM matrix was measured. As a positive control of biocompatibility, the proliferation of BAECs on type-1 collagen was also measured. At low density (1000 cells/cm²), medium density (5,000 cells/cm²), and high density (10,000 cells/cm²), there was no significant difference in proliferation on hAM matrix-coated surfaces, when compared to proliferation on Collagen-1 surfaces with similar cell densities (FIG. 3 A&B).

In addition, the cell biocompatibility of hAM matrix-coated surface was measured by live/dead cell staining at days 2 and 6 (FIG. 3C). The majority of ECs were alive during the cell culture period and became confluent after 6 days on hAM matrix. In support of this visual trend, the cell viability was measured by PrestoBlue® assay (FIG. 3D). Compared to commonly used collagen, no significant differences in proliferation, biocompatibility and viability were observed, suggesting that decellularized hAM matrix had excellent biocompatibility and biological activities for cell growth.

### Recovery of LV Function Following hAM Matrix Injection

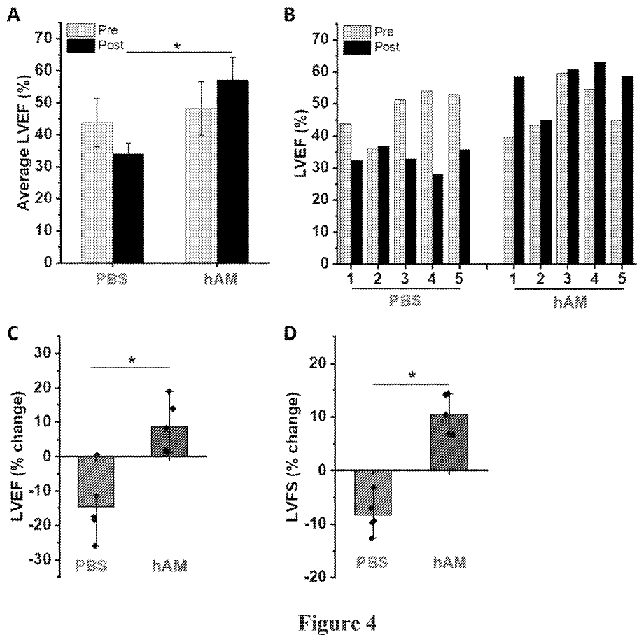

In vivo studies were performed by injecting hAM matrix into the acute infarcted myocardium. After 5 weeks following injection, hAM matrix-treated group showed significantly higher LVEF than PBS-treated group (57±7.1% for hAM vs 34±3.5% for PBS) (FIG. 4 A). Compared to the pre-treatment time point (Day 2 post-MI), the PBS-treated group generally demonstrated a continued decline in LV function (−9.7% worsening), while hAM matrix treatment prevented the negative LV remodeling and showed a significant improvement of LV function (+8.8%) (FIG. 4 B&C).

Fractional shortening also improved for animals receiving hAM matrix following acute MI (FIG. 4D). Improvements in fractional shortening were observed in all animals treated with hAM matrix injection. A decline in fractional shortening following acute MI was observed for all animals receiving PBS injection. On average, animals receiving hAM matrix injection experienced a +10.5% increase in fractional shortening, while animals receiving PBS injection experienced a −12.4% decline in fractional shortening.

### MI Size

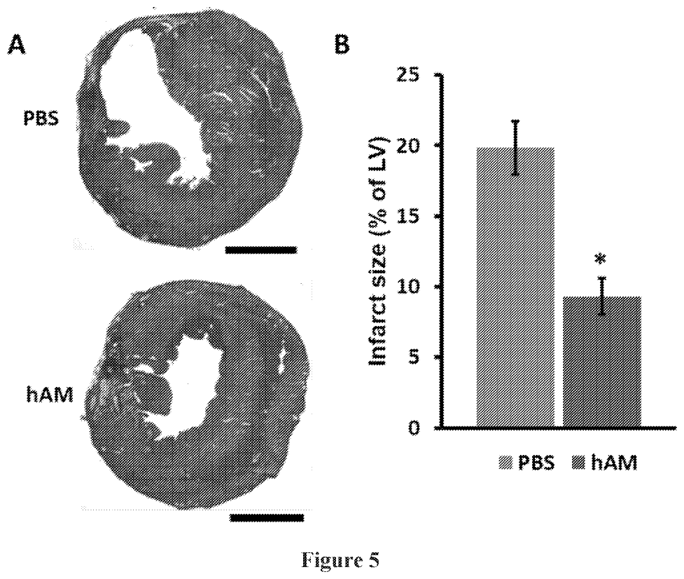

After 5 weeks, the MI rat hearts were harvested and cryosectioned for histological analyses. The cardiac fibrosis for hAM matrix or PBS treated hearts was evaluated with Masson's trichrome staining. Consistent with the functional analysis, there was a significant decrease in MI size as a percentage of the total LV in the hAM matrix treated group compared to the PBS group (FIG. 5). Infarct size was significantly reduced in the hAM matrix treated rats compared to the PBS treated rats (p<0.05).

### DISCUSSION

Heart failure following MI is a progressive process, consisting of several stages. During the acute phase, cardiomyocyte death occurs and is followed by macrophage, monocyte, and neutrophil migration. The inflammatory response continues through the subacute phase, until cellular components begin to be replaced by dense collagen fibrils. During the chronic phase, infarct expansion occurs, leading to dilation and continued LV remodeling.^{(37,38)} The injectable hydrogel, as a scaffold-based method, has shown great potential to treat MI by providing mechanical support and increasing myocardial thickness to prevent negative ventricular remodeling, which is advantageous as a minimally invasive and localized treatment⁽¹⁰⁾. For example, the use of alginate-based hydrogel, Algisyl-LVR, has shown encouraging preclinical results to improve myocardial functionality.^{(39,40)} The goal of this study was to develop an injectable ECM derived from human amniotic tissue and investigate its potential to promote recovery following myocardial infarction. To date, this study is the first to report the development of an injectable scaffold derived from decellularized amniotic membrane. Previous studies have utilized decellularization methods to fabricate injectable scaffolds from urinary bladder matrix, myocardial matrix, and lipoaspirate matrix.⁽⁴¹⁾ Our hypothesis is that hAM matrix may promote scarless wound healing similar to other fetal tissue, which allows regenerative remodeling. It is possible that some scar-inducing components in missing in hAM matrix or hAM matrix has anti-scar molecules, which needs further investigation.

The composition of ECM is unique to the organ/tissue from which it is derived. Likewise, decellularization methods must be tailored and made unique according to the tissue of interest. Through experimentation of various decellularization techniques, we have identified a process that is capable of preserving extracellular components, most specifically, the natural GAGs, from amniotic membrane. This process is highly efficient in removing cellular contents, as seen in the removal of greater than 96% of native DNA content. For future studies, it will be helpful to determine if higher levels of decellularization are needed to yield non-immunogenic matrix products.

Additionally, it is valuable to identify and quantify the protein components of the decellularized matrix by more advanced mass spectrum analysis to understand the material properties and biological effects.^{(42,43)}

Through in vitro cell studies on bovine aortic endothelium, we found that the injectable hAM matrix was non-cytotoxic and did not affect cell proliferation. In the future, it will be helpful to determine the effects on cell proliferation, attachment, and migration for other cardiac-specific cell lines. In addition, it will be helpful to determine if the extracted hAM matrix is capable of affecting myofibroblast differentiation, an event central to the process of scar formation.

To investigate the effect of hAM matrix injection on functional LV recovery following myocardial infarction, an acute rodent rat MI model was applied. We found that hAM matrix injection contributed to a reduction in MI scar formation and an improvement in LV function. Significant LV ejection fraction improvement was seen when each animal was used as its own control and with the hAM matrix group was compared to the control group. Due to the diversity in physiological response across various stages after MI, we chose to examine the effects of hAM matrix injection on cardiac function during the acute MI, 2 days after an MI. Hydrogel injection at 2-3 days post-MI has been commonly performed for acute MI therapy as a model to demonstrate therapeutic effects.^{(36, 44)} In the future, it will be valuable to evaluate the therapeutic effects of hAM matrix in chronic MI models, which will facilitate the translation of this technology into clinical applications.

Future studies can further elucidate the potential mechanism of LV functional improvement from hAM matrix injection for example comparisons of embodiments of the inventions to the effects of other matrix materials. By adding additional matrix groups to the study, it can be confirmed for example, whether LV functional improvement from hAM matrix injection is due to the unique biochemical environment of the amniotic membrane, or if LV improvement is due to a mechanical constrainment of LV dilation.

TABLE 1
Summary of major matrix proteins in injectable hAM matrix

| | Proteins |
|---|---|
| 1 | Collagen I |
| 2 | Collagen III |
| 3 | Collagen IV |
| 4 | Collagen V |
| 5 | Collagen VI |
| 6 | Collagen VII |
| 7 | Collagen XII |
| 8 | Collagen XIV |
| 9 | Collagen XVIII |
| 10 | Collagen XXVII |
| 11 | Fibronectin |
| 12 | Laminin |
| 13 | Keratin |
| 14 | Fibrillin-1 |
| 15 | Fibrillin-2 |
| 16 | Insulin-like Growth Factor Binding Protein |
| 17 | Latent TGF-β Binding Protein-1 |
| 18 | FGF Receptor-III |
| 19 | VEGF Receptor-I |

### REFERENCES

(1) Cahill T J, Choudhury R P, Riley P R, Nat. Rev. Drug Discov. 2017, 16, 699.
(2) Kim G H, Uriel N, Burkhoff D, Nat. Rev. Cardiol. 2018, 15, 83.
(3) Tallquist M D, Molkentin J D, Nat. Rev. Cardiol. 2017, 14, 484.
(4) Hashimoto H, Olson E N, Bassel-Duby R, Nat. Rev. Cardiol. 2018, 15, 585.
(5) Parsa H, Ronaldson K, Vunjak-Novakovic G, Adv. Drug Deliver. Rev. 2016, 96, 195.
(6) Mahmoudi M, Yu M, Serpooshan V, Wu J C, Langer R, Lee R T, Karp J M, Farokhzad O C, Nat. Nanotechnol. 2017, 12, 845.
(7) Gourdie R G, Dimmeler S, Kohl P, Nat. Rev. Drug Discov. 2016, 15, 620.
(8) Chen W C W, Wang Z G, Missinato M A, Park D W, Long D W, Liu H J, Zeng X M, Yates N A, Kim K, Wang Y D, Sci. Adv. 2016, 2.
(9) Becker M, Maring J A, Schneider M, Herrera Martin A X, Seifert M, Klein O, Braun T, Falk V, Stamm C, Int. J. Mol. Sci. 2018, 19.
(10) Hasan A, Khattab A, Islam M A, Abou Hweij K, Zeitouny J, Waters R, Sayegh M, Hossain M M, Paul A, Adv. Sci. 2015, 2.
(11) Bejleri D, Davis M E, Adv. Healthc. Mater. 2019, 8, 1801217.
(12) Jin Y, Lee J S, Kim J, Min S, Wi S, Yu J H, Chang G E, Cho A N, Choi Y, Ahns D H, Cho S R, Cheong E, Kim Y G, Kim H P, Kim Y, Kim D S, Kim H W, Quan Z, Kang H C, Cho S W, Nat. Biomed. Eng. 2018, 2, 522.
(13) Taylor D A, Sampaio L C, Ferdous Z, Gobin A S, Taite L J, Acta Biomater. 2018, 74, 74.
(14) Yu C, Ma X, Zhu W, Wang P, Miller K L, Stupin J, Koroleva-Maharajh A, Hairabedian A, Chen S, Biomaterials 2019, 194, 1.
(15) Ma X Y, Yu C, Wang P R, Xu W Z, Wan X Y, Lai C S E, Liu J, Koroleva-Maharajh A, Chen S C, Biomaterials 2018, 185, 310.
(16) Ghuman H, Mauney C, Donnelly J, Massensini A R, Badylak S F, Modo M, Acta Biomater. 2018, 80, 66.
(17) Zhu Y, Hideyoshi S, Jiang H B, Matsumura Y, Dziki J L, LoPresti S T, Huleihel L, Faria G N F, Fuhrman L C, Lodono R, Badylak S F, Wagner W R, Acta Biomater. 2018, 73, 112.
(18) Hussey G S, Keane T J, Badylak S F, Nat. Rev. Gastro. Hepat. 2017, 14, 540.
(19) Moroni F, Mirabella T, Am. J. Stem Cells 2014, 3, 1.
(20) Saldin L T, Cramer M C, Velankar S S, White L J, Badylak S F, Acta. Biomater. 2017, 49, 1.
(21) Hariya T, Tanaka Y, Yokokura S, Nakazawa T, Biomaterials 2016, 101, 76.
(22) Ben M'Barek K, Habeler W, Plancheron A, Jarraya M, Regent F, Terray A, Yang Y, Chatrousse L, Domingues S, Masson Y, Sahel J A, Peschanski M, Goureau O, Monville C, Sci. Transl. Med. 2017, 9.
(23) Despeyroux A, Duret C, Gondeau C, Perez-Gracia E, Chuttoo L, de Boussac H, Briolotti P, Bony C, Noel D, Jorgensen C, Larrey D, Daujat-Chavanieu M, Herrero A, J. Tissue Eng. Regen. M. 2018, 12, 1062.
(24) Stern M, J. Amer. Med. Assoc. 1913, 60, 973.
(25) Shimmura S, Shimazaki J, Ohashi Y, Tsubota K, Cornea 2001, 20, 408.
(26) Hashim S N M, Yusof M F H, Zahari W, Noordin K B A A, Kannan T P, Hamid S S A, Mokhtar K I, Ahmad A, Tissue Eng. Regen. Med. 2016, 13, 211.
(27) Navas A, Magana-Guerrero F S, Dominguez-Lopez A, Chavez-Garcia C, Partido G, Graue-Hernandez E O, Sanchez-Garcia F J, Garfias Y, Stem Cell Transl Med. 2018, 7, 906.
(28) Niknejad H, Peirovi H, Jorjani M, Ahmadiani A, Ghanavi J, Seifalian A M, Eur. Cells Mater. 2008, 15, 88.
(29) Farndale R W, Buttle D J, Barrett A J, Biochim. Biophys. Acta 1986, 883, 173.
(30) Lee J Y, Kim H, Ha D H, Shin J C, Kim A, Ko H S, Cho D W, Adv. Healthc. Mater. 2018, 7, e1800673.
(31) Li S, Kim M, Hu Y L, Jalali S, Schlaepfer D D, Hunter T, Chien S, Shyy J Y, J. Biol. Chem. 1997, 272, 30455.
(32) Christman K L, Fok H H, Sievers R E, Fang Q, Lee R J, Tissue Eng. Part B-Re. 2004, 10, 403.
(33) Huang N F, Sievers R E, Park J S, Fang Q, Li S, Lee R J, Nat. Protoc. 2006, 1, 1596.
(34) Mihardja S S, Gonzales J A, Gao D, Sievers R E, Fang Q, Stillson C A, Yu J, Peng M, Lee R J, Biomaterials 2013, 34, 8869.
(35) Yu J S, Christman K L, Chin E, Sievers R E, Saeed M, Lee R J, J. Thorac. Cardiov. Sur. 2009, 137, 180.
(36) Le L V, Mohindra P, Fang Q Z, Sievers R E, Mkrtschjan M A, Solis C, Safranek C W, Russell B, Lee R J, Desai T A, Biomaterials 2018, 169, 11.
(37) Anderson J L, Morrow D A, N. Engl. J. Med. 2017, 376, 2053.
(38) Hausenloy D J, Yellon D M, Nat. Rev. Cardiol 2011, 8, 619.
(39) Lee L C, Wall S T, Klepach D, Ge L, Zhang Z H, Lee R J, Hinson A, Gorman J H, Gorman R C, Guccione J M, Int. J. Cardiol. 2013, 168, 2022.
(40) Lee R J, Hinson A, Bauernschmitt R, Matschke K, Fang Q, Mann D L, Dowling R, Schiller N, Sabbah H N, Int. J. Cardiol. 2015, 199, 18.
(41) Johnson T D, Braden R L, Christman K L, Methods Mol. Biol. 2014, 1181, 109.
(42) Zhang X, Fang A, Riley C P, Wang M, Regnier F E, Buck C, Anal. Chim. Acta 2010, 664, 101.
(43) Johnson T D, Hill R C, Dzieciatkowska M, Nigam V, Behfar A, Christman K L, Hansen K C, Proteomics Clin. Appl. 2016, 10, 75.
(44) Yoshizumi T, Zhu Y, Jiang H, D'Amore A, Sakaguchi H, Tchao J, Tobita K, Wagner W R, Biomaterials 2016, 83, 182.

All publications mentioned herein (e.g., Henry et al., Advanced Healthcare Materials. 2020 January; 9(2): e1900544, and those identified above) are incorporated by reference to disclose and describe aspects, methods and/or materials in connection with the cited publications. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

1. A method of making a human amniotic membrane (hAM) hydrogel matrix composition, the method comprising:
combining a human amniotic membrane with agents selected to remove cells and nucleic acids so as to form a decellularized human amniotic membrane;
rinsing and/or dialyzing the decellularized human amniotic membrane so as to remove agents selected to remove cells and nucleic acids;
lyophilizing the rinsed and/or dialyzed decellularized human amniotic membrane;
forming a dry powder from the lyophilized human amniotic membrane;
solubilizing the dry powder to form a solution;
adjusting the pH of the solubilized dry powder solution; and
lyophilizing the pH adjusted solubilized dry powder solution, so that the human amniotic membrane (hAM) hydrogel matrix powder composition is formed.

2. The method of claim 1, further comprising resuspending the human amniotic membrane hydrogel matrix powder composition in an aqueous solution so as to form an injectable human amniotic membrane (hAM) hydrogel matrix composition.

3. The method of claim 2, wherein the injectable human amniotic membrane hydrogel matrix composition:
forms a liquid at temperatures from 0° C. to 20° C.; and
forms a gel at 37° C.

4. The method of claim 2, wherein the injectable human amniotic membrane (hAM) hydrogel matrix composition further comprises:
a pharmaceutical excipient selected from the group consisting of: a preservative, a tonicity adjusting agent, a detergent, a viscosity adjusting agent, a sugar and a pH adjusting agent;
a therapeutic agent such as an anti-fibrotic agent, an anti-inflammatory agent, a hemostatic agent, or a chemotherapeutic agent; and/or
mammalian cells.

5. The method of claim 1, wherein the human amniotic membrane (hAM) hydrogel matrix composition is formed to comprise:
a glycosoaminoglycan content that is at least 10%, 20%, 30%, 40% 50%, 60%, 70% or 80% of the glycosoaminoglycan content of the native human amniotic membrane; and/or
a collagen content that is at least 10%, 20%, 30%, 40% or 45% of the collagen content of the native human amniotic membrane.

6. A composition formed by the method of claim 1.

7. A composition of matter comprising a human amniotic membrane (hAM) hydrogel, wherein:
the human amniotic membrane comprises a glycosoaminoglycan content comprising from about 70% to about 90% of the glycosoaminoglycan content found in native human amniotic membranes;
the human amniotic membrane comprises a collagen content comprising from about 30% to about 60% of the collagen content found in native human amniotic membranes; and
the human amniotic membrane comprises less than 10% of the DNA content found in native human amniotic membranes.

8. The composition of claim 7, wherein the human amniotic membrane (hAM) hydrogel composition forms a liquid at temperatures from 0° C. to 20° C.; and forms a gel at 37° C.

9. The composition of claim 6 wherein:
the composition exhibits a shear modulus of about 7.5±2.4 Pa as determined by oscillatory rheometry; and/or
at 1% strain amplitude, the composition exhibits a storage modulus of 1 PA-20 kPA at a frequency of about 1 Hz.

10. A method of delivering a liquid composition of claim 6 to a preselected site comprising:
disposing the composition in a vessel having a first end comprising an opening and a second end;
applying a force to the second end of the vessel, wherein the force is sufficient to force the liquid through the first end of the vessel; and
delivering the composition out of the vessel through the opening and to the preselected site.

11. The method of claim 10, wherein the site is an in vivo site.

12. The method of claim 11, wherein the site is at an in vivo location where an individual has experienced cardiac injury.

13. The method of claim 11, wherein the site is at an in vivo location within the cardiac tissue within or near the injured cardiac injury.

14. A method of inhibiting fibrosis at a site of cardiac injury in an individual, the method comprising: disposing the composition of claim 6 at the site of the cardiac injury such that the composition modulates cardiac remodeling, so that fibrosis is inhibited.

15. The method of claim 14, wherein cardiac remodeling comprises:
an inhibition of negative ventricular remodeling; and/or
a decrease in myocardial infarction size.

16. The method of claim 14, wherein the composition:
forms a liquid at temperatures from 0° C. to 20° C.; and
forms a gel at 37° C.

17. The method of claim 14, wherein the composition comprises:
a pharmaceutical excipient selected from the group consisting of: a preservative, a tonicity adjusting agent, a detergent, a viscosity adjusting agent, a sugar and a pH adjusting agent;
a therapeutic agent such as an anti-fibrotic agent, an anti-inflammatory agent, a hemostatic agent, or a chemotherapeutic agent; and/or
mammalian cells.

18. The method of claim 14, wherein:
the composition exhibits a shear modulus of about 7.5±2.4 Pa as determined by oscillatory rheometry; and/or
at 1% strain amplitude, the composition exhibits a storage modulus of 1 PA-20 kPA at a frequency of about 1 Hz.

19. The method of claim 14, wherein the human amniotic membrane hydrogel matrix comprises a glycosoaminoglycan content that is not more than 90% of the glycosoaminoglycan content of a native human amniotic membrane.

20. The method of claim 19, wherein the human amniotic membrane hydrogel matrix comprises a collagen content that is not more than 50% of the collagen content of the native human amniotic membrane.
